(19) United States
(12) Patent Application Publication
(10) Pub. No.: US 2022/0304576 A1
(43) Pub. Date: Sep. 29, 2022

(54) Remote Health Monitoring System and Method

(71) Applicant: David Edward Kiger, Knoxville, TN (US)

(72) Inventor: David Edward Kiger, Knoxville, TN (US)

(21) Appl. No.: 17/704,603

(22) Filed: Mar. 25, 2022

Related U.S. Application Data
(60) Provisional application No. 63/165,776, filed on Mar. 25, 2021.
(60) Provisional application No. 63/247,065, filed on Sep. 22, 2021.

(51) Int. Cl. A61B 5/00; A61B 5/021; A61B 5/1455

(52) U.S. Cl. CPC A61B 5/0022; A61B 5/0004; A61B 5/02141; A61B 5/14552

(57) ABSTRACT

A system and method to remotely monitor the health of a patient, the system including a tablet device preconfigured to allow a predetermined number of applications to be operated by a designated user, and one or more health data collecting devices preconfigured to communicate with the tablet device so as to transmit collected data to the tablet device, wherein at least one of the applications of the tablet device is a health data application configured such that a user can begin a health data collecting procedure with by opening the health data application and operating one of the health data collecting devices, and wherein the tablet device is configured to transmit the collected data to a remotely located health care administration system.

### CROSS-REFERENCE TO RELATED APPLICATIONS

This application claims the benefit of U.S. Provisional Patent Application Ser. No. 63/165,776, filed on Mar. 25, 2021, and U.S. Provisional Patent Application Ser. No. 63/247,065, filed on Sep. 22, 2021, the contents of both of which are incorporated herein in their entirety by reference.

### FIELD OF INVENTION

The present general inventive concept relates to a health monitoring system, and, more particularly, to a remote health monitoring system designed for ease of use by senior citizens.

### BACKGROUND

Affordable and convenient health monitoring is a concern for a growing segment of our population such as, for example, senior citizens. Since many health problems can be less threatening when detected and treated early, but so many people are underinsured, it is a source of anxiety for many as to when to try and have even routine checkups performed, much less more involved diagnostic tests. This has become even more problematic in recent times, when pandemic fears have people concerned about even leaving their homes for any medical evaluation. Modern technology has developed a variety of mobile and other such devices that increase the convenience of communication and various type of information processing, but a large segment of the elderly population may be either unfamiliar with such technology, and/or uncomfortable with using such devices. Thus, there exists a need for a convenient way to assess various health metrics without the patient having to leave the home, and in a simple and convenient process that most anyone can navigate.

### BRIEF SUMMARY

According to various example embodiments of the present general inventive concept, a method of remote health care is provided which allows a patient to facilitate collection of various medical/health data to be sent to a healthcare provider through an information processing device such as a tablet, presenting the health data to the healthcare provider, and establishing an audio/video communication feed between the patient and the healthcare provider.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by providing a system to remotely monitor the health of a patient, the system including a tablet device preconfigured to allow a predetermined number of applications to be operated by a designated user, and one or more health data collecting devices preconfigured to communicate with the tablet device so as to transmit collected data to the tablet device, wherein at least one of the applications of the tablet device is a health data application configured such that a user can begin a health data collecting procedure with by opening the health data application and operating one of the health data collecting devices, and wherein the tablet device is configured to transmit the collected data to a remotely located health care administration system.

The foregoing and/or other aspects and advantages of the present general inventive concept may also be achieved by providing a method of remote monitoring of the health of a patient, the method including providing a system to a user that is configured to associated with health data of the user, the system including a tablet device preconfigured to allow a predetermined number of applications to be operated by a designated user, and one or more health data collecting devices preconfigured to communicate with the tablet device so as to transmit collected data to the tablet device, wherein at least one of the applications of the tablet device is a health data application configured such that a user can begin a health data collecting procedure with by opening the health data application and operating one of the health data collecting devices, and wherein the tablet device is configured to transmit the collected data to a remotely located health care administration system.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates remote health monitoring system according to an example embodiment of the present general inventive concept;

FIG. 2 illustrates a display on the tablet of FIG. 1 according to an example embodiment of the present general inventive concept;

FIG. 3 illustrates a health data collection operation using the system 100 illustrated in FIG. 1;

FIG. 4 illustrates a direct video communication between a patient and caregiver using the remote health care monitoring system according to an example embodiment of the present general inventive concept;

FIG. 5 illustrates a process in which different types of devices monitor and transmit a wide variety of medical data through public application programming interfaces (API's) to a data processing entity according to an example embodiment of the present general inventive concept;

FIG. 6 is a flow chart illustrating an operation of compiling health data from multiple sources, and then reorganizing the data so as to be presented on one screen according to an example embodiment of the present general inventive concept; and

FIG. 7 illustrates information that may be available in a patient screen view according to an example embodiment of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

According to various example embodiments of the present general inventive concept, a system or “kit” can be provided to a patient that includes a pre-programmed information processing device with a screen, which may be referred to generically as a “tablet” in the descriptions herein, which provides a limited number of easily identifiable and accessible processes to a patient by touching one of the buttons on the screen. Thus, since many people, especially among the elderly, may be unfamiliar or unwilling to become overly familiar with typical screen devices, the present general inventive concept may provide such users with an easy way to check various health measurements, such as one or more vital signs, at home, and have that information transmitted to a health care provided with a simple push of a button on the table. In various example embodiments, the patient receives the tablet as part of a kit or system that may include one or more measurement devices to measure or monitor one or more vital signs or other health indicators. Since the tablet may be preprogrammed, in various example embodiments the patient does not have to create an account or password, or download an app, or other things that can be extremely frustrating to people that do not typically use such devices. The table may come to the patient via mail, and may be preprogrammed to that particular patient, and may allow the collection of health measurements in conjunction with a provided device by a simple button on the screen, and those measurements may be automatically transmitted to a health care provider/system that monitors the measurements for signs of trouble.

Various example embodiments of the present general inventive concept provide a healthcare system in which an information processing device with a screen, such as a tablet, is given to the client/patient. Such a device may be provided at no charge to the patient. All other information, software, etc., on the table may have been removed, or may be “hidden” from the patient through security locks or the like. The healthcare system software may be installed on the tablet and may control the table such that only a specific number of buttons, e.g., three buttons, are visible on the tablet for the patient to use as an interface. In this way, there is no clutter or otherwise confusing display information to hinder the patient's use of the tablet. In various example embodiments the tablet may be provided with one or more devices such as, for example, a blood pressure cuff that is already arranged to communicate with the tablet, in a “kit” for the patient. All information gathered by using the device may be sent back to one designated provider. The tablet may be provided with Telehealth code written to conduct a live audio/video feed from the tablet to the medical professional on the other end. Physician specific software may already be paired to devices such as, for example, a blood pressure cuff, blood glucose tester, oxygen sensor, weight scale, etc., or any combination thereof. Such devices may be referred to generically as health data collection devices in the descriptions herein, and may be configured to transmit the gathered health data wirelessly, and/or through a wired connection, to the tablet. This information may be automatically uploaded through a web connection for the specified medical professional to view. An IT button may be added for someone to choose if they are having trouble with any of the equipment. The end product is a preprogrammed health tablet delivering the patient data to their own specific health professional, not a stranger like other conventional devices. If that health data indicates that attention may be needed, a telehealth visit is initiated again, by one specific health professional or office.

The devices used to gather or collect medical/health data to be transmitted to the physician may be dedicated devices, such as, for example, a blood pressure cuff or thermometer or weight scale, etc., that communicate health data to the tablet, or more general devices like a smart watch that monitors pulse and movement activity and so on.

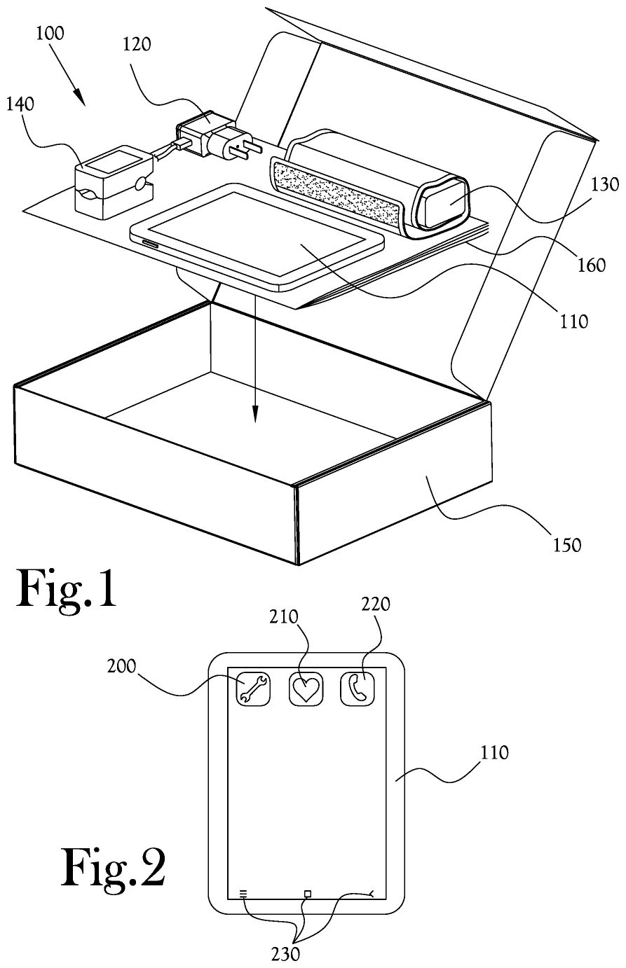

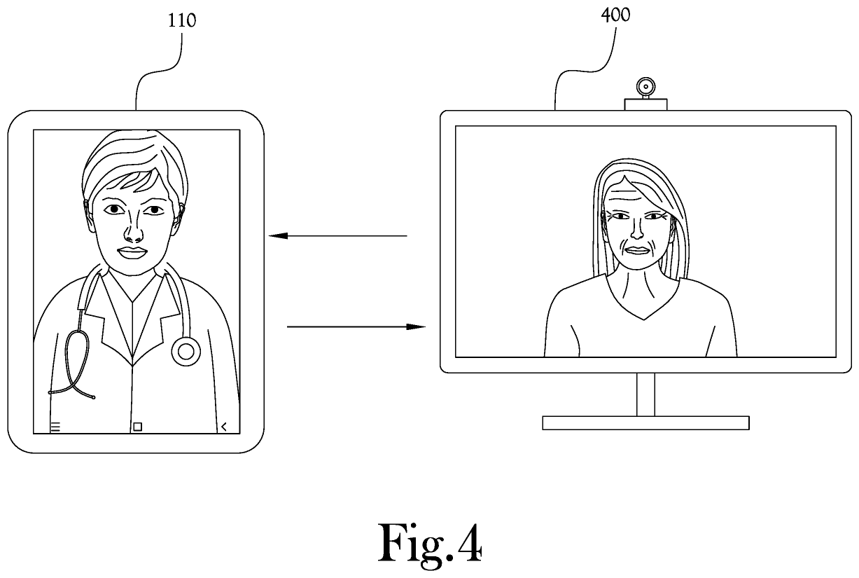

FIG. 1 illustrates remote health monitoring system according to an example embodiment of the present general inventive concept. a tablet that may be used by the patient. As illustrated in FIG. 4, the remote heath monitoring system 100 includes a tablet 110, a power cord 120 for charging the tablet, a blood pressure cuff 130 equipped with a wireless controller/transmitter, and a fingertip pulse oximeter 140 that have been bundled together in a box 150 with corresponding literature/manuals 160 associated with the system and operation thereof. It is understood that the devices illustrated in this system are merely examples of various devices that may be included, and more or fewer and/or different devices may be included without departing from the scope of the present general inventive concept. By providing this system 100 in a kit such as shown, it is very convenient for the patient to be able to operate the associated equipment, and may even use the box 150 to maintain storage of the entire system. The blood pressure cuff 130 and pulse oximeter 140 have been previously paired with the tablet 110 so that the system is ready to go right out of the box, with no painful and time-consuming setup or registration procedures to be performed by the user.

FIG. 2 illustrates a display on the tablet 110 of FIG. 1 according to an example embodiment of the present general inventive concept. The screen of the tablet may display a remote care communication “button” 220 which will facilitate an audio-video feed with the patient's health care provider, a medical data button 210 which will initiate the collecting of the medical/health data from the patient, and an IT button 200 which will initiate contact with an information technology aid to help with equipment trouble. Thus, with only three selectable buttons it is very easy for a user to see what is available, and how to choose from the available options. In various example embodiments, the buttons may be displayed with easily identifiable indicia to indicate their purpose. For example, the IT button 200 may display a wrench, the medical data button 210 may display a heart, or a red cross, etc., and the remote care communication button 220 may display a telephone of the like. The buttons may also have text on the buttons and/or below the buttons to further identify their functions. Various typical tablet controls 230 may be displayed at the bottom of the screen for various tablet navigation/operations. Thus, for example, if the patient/user wishes to gather medical data, the patient can simply choose the medical data button 210, which opens the data collection app that will automatically pair and receive data from any associated device being used by the patient, such as that blood pressure cuff 130 of FIG. 1. The medical data may be any of a host of types of data, such as blood pressure, blood glucose levels, blood oxygen levels, temperature, pulse, weight, and so on. In various example embodiments the traditional vital signs may be automatically prompted to the patient and/or uploaded to the tablet 110. The tablet may then transmit any and all of the medical data to a remote health care provider/system that monitors the data for changes that may require closer monitoring or immediate attention.

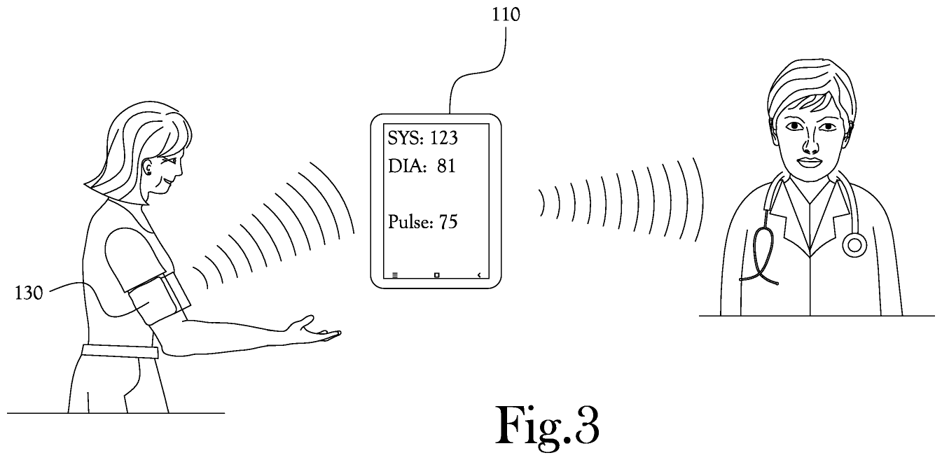

FIG. 3 illustrates a health data collection operation using the system 100 illustrated in FIG. 1. As illustrated in FIG. 3, a patient has pressed the medical data button 210 on the tablet 110 and applied the blood pressure cuff 130 to her arm. The blood pressure cuff 130, which was previously paired with the tablet 110 before being received by the patient, sends measured health data to the table 110, which is displayed on the screen, and the tablet 110 in turn sends the data to a health care provider and/or system. In various example embodiments of the present general inventive concept, the tablet 110 may be configured to send a priority alert to a third party or system if any of the measured health data is outside a predetermined parameter, such as sending a high priority message to a designated health care provider associated with the system.

In various example embodiments the measured data may be displayed on the tablet 110 through the medical data app, and if the patient is concerned about any of the information the patient can press the communication button 220 to initiate a face to face conversation with a health care provider. FIG. 4 illustrates a direct video communication between a patient and caregiver using the remote health care monitoring system according to an example embodiment of the present general inventive concept. As illustrated, the patient is able to view the caregiver on the tablet 110, and the caregiver is able to view the patient on a screen used by the caregiver, which in this example embodiments is a computer screen 400. Thus, such face to face communication is available so that a caregiver can further assess a medical situation, provide instructions for further diagnostics, etc., and the patient can be reassured by seeing the face of the doctor, which recreates the feel of an actual face to face visit in a doctor's office.

The devices for collecting the information may transmit the information through a wireless connection such as Bluetooth, or may have wired connections to the tablet. In various example embodiments multiple buttons that are dedicated to various types of medical data may be displayed, but the single medical data button may be especially convenient for seniors or other people that are not as “computer literate” as others. Thus, a system 100 is provided that includes a simplified interface with which a patient can interact to gather or collect various medical data that can be sent nearly instantly to a physician in an organized and sometimes prioritized manner, so that the physician or other healthcare provider can quickly evaluate the data and initiate immediate contact with the patient if necessary. With such a system, the patient's health can be monitored in a meaningful way without the patient having to leave his or her home, and health problems can be recognized at treated early on. Thus, the virtual care system provided by various example embodiments of the present general inventive concept may cater specifically to elderly and technologically challenged users. The system may be cellular based to prevent issues with Wi-Fi or other connectivity. Various example embodiments of the system may include devices to measure heart rate, heart rhythm, oxygen, glucose, weight, and more, combined with a built-in virtual visit platform that helps caregivers and practitioners provide preventative care and keep high-risk individuals out of the emergency room. Systems configured according to the present general inventive concept may save both the patients and caregivers time, reduce practice-wide administrative time and paperwork, reduce readmissions into the hospitals, etc. A remote health care administrative system may assign and customize all of the equipment in the systems or kits sent to the individual patients, and the physician's office can immediately and remotely monitor all readings and may receive, for example, color-coded alerts highlighting patient needs. The medical staff may reach out to the patient as necessary, and, if necessary, may schedule a telehealth visit.

According to various example embodiments of the present general inventive concept, a method and system is provided in which a series of processes may be utilized to collect statistical data, which may include non-medical data, from a wide variety of devices, which may be from a wide variety of manufacturers, to combine all of the data into one program in order to send it to one display screen (primarily for the physician) to evaluate, while also sending alerts in order to monitor the data readings. The screen presented to the physician will not only show the statistical data but also allow for real time audio and video interfacing between a patient and the physician in order to discuss the data. Various example embodiments of the present general inventive concept may be especially convenient in assisting senior citizens, but may be used in any person's health monitoring situation.

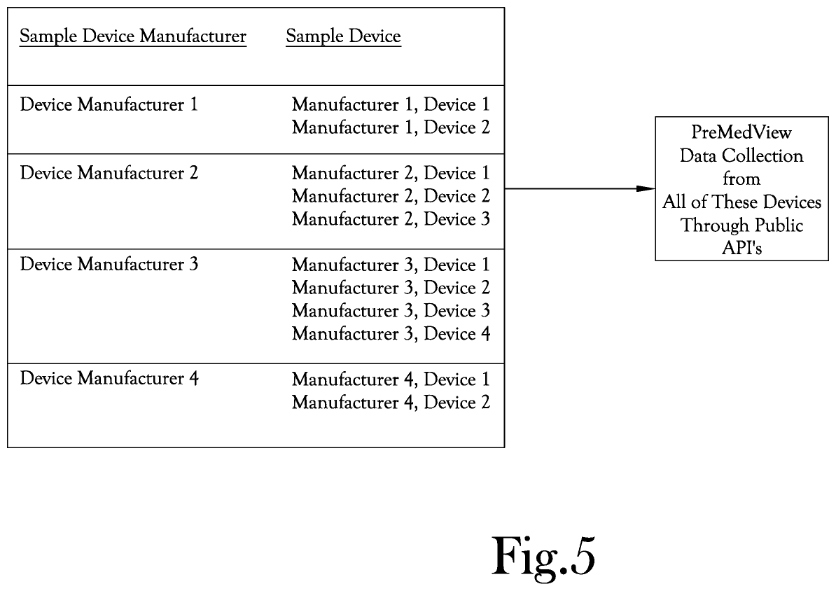

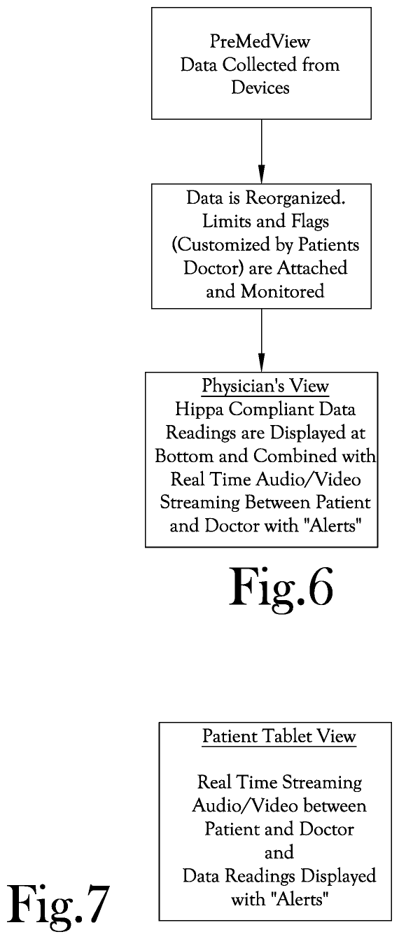

FIG. 5 illustrates a process in which different types of devices monitor and transmit a wide variety of medical data through public application programming interfaces (API's) to a data processing entity, identified as PreMedView in FIG. 5, according to an example embodiment of the present general inventive concept. As illustrated, a host of manufacturers' devices may be utilized, as well as a host of the devices, to collect such information as weight, blood pressure, temperature, other vital signs, etc. All such data may be collected through the public (API's) or directly from the device or tablet in the home via a wireless connection such as BLUETOOTH®, and as illustrated in FIG. 6, may be further processed by the data processing entity. FIG. 6 is a flow chart illustrating an operation of compiling the data from multiple sources, and then reorganizing the data so as to be presented on one screen with simple tabs at the bottom. The data may also be analyzed such that limits and flags are attached for monitoring purposes. The data is then displayed on the physician's screen, and selective audio/video capability is enabled between the patient and the physician. FIG. 7 illustrates information that may be available in a patient screen view, on a device such as a tablet. In this example view the data has been compiled and reorganized onto one screen with simple tabs at the bottom. The data may be analyzed, and limits and flags may be attached for monitoring purposes. The data may then be displayed on the physician screen, and the audio/video capability may be enabled between the patient and the physician. When an audio/video chat occurs between the patient and the physician, various example embodiments of the system may be programmed to convert the audio portion to text (a “chat to text” feature), and to email or otherwise transmit the transcript to an account manager listed on the customer profile.

Various example embodiments of the present general inventive concept are designed to collect data from multiple devices and manufacturers using public API's, BLUETOOTH®, etc. That data is then put into a monitoring program with limits and flags and individual patient and doctor information. Alerts may then be sent when one of the limits or flags have been breached. The physician can then see these flags, along with all of the data that has been collected, and can immediately initiate an audio/video conference with the patient and vice versa. This immediate access to a physician will change the level of care for senior citizens, providing timely and relevant data such that the physician can make an informed decision for prognoses and diagnoses. Each manufacturer of the various information collecting devices already has developed app's for their individual devices. The system of the present general inventive concept can collect this data from all preapproved (e.g., by insurers) devices, after the devices are selected by the physician and the patient. Thus, various example embodiments of the present general inventive concept collect data from multiple public data sources, reorganize that data, add alerts, and combine with real time audio/video through new software in order to make available a new elevated level of telehealth care to the industry. The system may collect data from various sources, including new sources and devices coming online every day, and import it into the system's software. The data may then be combined with alerts and real time audio/video software. This software may then be made available to patients and their physicians for preventative telehealth care. This enables the first software for a “Virtual Doctor Visit.”

Various example embodiments of the present general inventive concept may provide a system to remotely monitor the health of a patient, the system including a tablet device preconfigured to allow a predetermined number of applications to be operated by a designated user, and one or more health data collecting devices preconfigured to communicate with the tablet device so as to transmit collected data to the tablet device, wherein at least one of the applications of the tablet device is a health data application configured such that a user can begin a health data collecting procedure with by opening the health data application and operating one of the health data collecting devices, and wherein the tablet device is configured to transmit the collected data to a remotely located health care administration system. At least one of the applications of the tablet device may be a communication application configured to initiate an audio and visual communication with a designated health care administrator when the user opens the communication application. At least one of the applications of the tablet device may be a technical support application configured to initiate a communication with a designated technical support administrator when the user opens the technical support application. The tablet device may be configured such that no further applications can be added to the tablet device except by a user. The one or more health collecting devices may include a blood pressure cuff configured with a wireless controller/transmitter to collect blood pressure related health data to be transmitted to the tablet device. The one or more health collecting devices may also include a pulse oximeter configured to collect health data to be transmitted to the tablet device. The system may be configured as a kit to be delivered to a user such that the user can use the system immediately upon powering the tablet device.

Various example embodiments of the present general inventive concept may provide a method of remote monitoring of the health of a patient, the method including providing a system to a user that is configured to associated with health data of the user, the system including a tablet device preconfigured to allow a predetermined number of applications to be operated by a designated user, and one or more health data collecting devices preconfigured to communicate with the tablet device so as to transmit collected data to the tablet device, wherein at least one of the applications of the tablet device is a health data application configured such that a user can begin a health data collecting procedure with by opening the health data application and operating one of the health data collecting devices, and wherein the tablet device is configured to transmit the collected data to a remotely located health care administration system. At least one of the applications of the tablet device may be a communication application configured to initiate an audio and visual communication with a designated health care administrator when the user opens the communication application. At least one of the applications of the tablet device may be a technical support application configured to initiate a communication with a designated technical support administrator when the user opens the technical support application. The tablet device may be configured such that no further applications can be added to the tablet device except by a user. The one or more health collecting devices may include a blood pressure cuff configured with a wireless controller/transmitter to collect blood pressure related health data to be transmitted to the tablet device. The one or more health collecting devices may also include a pulse oximeter configured to collect health data to be transmitted to the tablet device. The system provided in this method may be configured as a kit to be delivered to a user such that the user can use the system immediately upon powering the tablet device.

Various example embodiments of the present general inventive concept may provide a method of evaluating the health of a remotely located patient, the method including providing an information processing device to a patient, gathering health data of the patient through the information processing device, and transmitting the health data to a healthcare provider for evaluation. The health data may be obtained by one or more devices in the patient's home. The information processing device may be a tablet with three buttons displayed, the buttons actuating contact with the healthcare provider, collection of the health data, and contact with I.T. support, respectively. The method may further include organizing and analyzing the health data before presenting the health data to the healthcare provider. The method may further include flagging at least a portion of the health data to indicate urgent attention being needed. The method may further include establishing a direct audio and video feed between the patient and the physician to discuss the health data.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering judgment.

Numerous variations, modification, and additional embodiments are possible, and, accordingly, all such variations, modifications, and embodiments are to be regarded as being within the spirit and scope of the present general inventive concept.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

1. A system to remotely monitor the health of a patient, the system comprising:
a tablet device preconfigured to allow a predetermined number of applications to be operated by a designated user; and
one or more health data collecting devices preconfigured to communicate with the tablet device so as to transmit collected data to the tablet device;
wherein at least one of the applications of the tablet device is a health data application configured such that a user can begin a health data collecting procedure with by opening the health data application and operating one of the health data collecting devices; and
wherein the tablet device is configured to transmit the collected data to a remotely located health care administration system.

2. The system of claim 1, wherein at least one of the applications of the tablet device is a communication application configured to initiate an audio and visual communication with a designated health care administrator when the user opens the communication application.

3. The system of claim 1, wherein at least one of the applications of the tablet device is a technical support application configured to initiate a communication with a designated technical support administrator when the user opens the technical support application.

4. The system of claim 1, wherein the tablet device is configured such that no further applications can be added to the tablet device except by a user.

5. The system of claim 1, wherein the one or more health collecting devices includes a blood pressure cuff configured with a wireless controller/transmitter to collect blood pressure related health data to be transmitted to the tablet device.

6. The system of claim 5, wherein the one or more health collecting devices also includes a pulse oximeter configured to collect health data to be transmitted to the tablet device.

7. The system of claim 1, configured as a kit to be delivered to a user such that the user can use the system immediately upon powering the tablet device.

8. A method of remote monitoring of the health of a patient, the method comprising:
providing a system to a user that is configured to associated with health data of the user, the system comprising:
a tablet device preconfigured to allow a predetermined number of applications to be operated by a designated user, and
one or more health data collecting devices preconfigured to communicate with the tablet device so as to transmit collected data to the tablet device;
wherein at least one of the applications of the tablet device is a health data application configured such that a user can begin a health data collecting procedure with by opening the health data application and operating one of the health data collecting devices; and
wherein the tablet device is configured to transmit the collected data to a remotely located health care administration system.

9. The method of claim 8, wherein at least one of the applications of the tablet device is a communication application configured to initiate an audio and visual communication with a designated health care administrator when the user opens the communication application.

10. The method of claim 8, wherein at least one of the applications of the tablet device is a technical support application configured to initiate a communication with a designated technical support administrator when the user opens the technical support application.

11. The method of claim 8, wherein the tablet device is configured such that no further applications can be added to the tablet device except by a user.

12. The method of claim 8, wherein the one or more health collecting devices includes a blood pressure cuff configured with a wireless controller/transmitter to collect blood pressure related health data to be transmitted to the tablet device.

13. The method of claim 12, wherein the one or more health collecting devices also includes a pulse oximeter configured to collect health data to be transmitted to the tablet device.

14. The method of claim 8, wherein the provided system is configured as a kit to be delivered to a user such that the user can use the system immediately upon powering the tablet device.
